(19) United States
(12) Patent Application Publication
(10) Pub. No.: US 2025/0306005 A1
(43) Pub. Date: Oct. 2, 2025

(54) MEANS AND METHODS FOR DETERMINING CELLULAR AVIDITY

(71) Applicant: LUMICKS CA HOLDING B.V., Amsterdam (NL)

(72) Inventors: Patrycja Anna WAWRZYNIECKA, Amsterdam (NL); Mirjam Rebekka DALENBERG, Amsterdam (NL); Trillian Ashley GREGG, Amsterdam (NL); Marinus Theodorus Johannes VAN LOENHOUT, Amsterdam (NL); Serena Alba DAVOLI, Amsterdam (NL); Mattijs DE GROOT, Amsterdam (NL)

(21) Appl. No.: 18/863,923

(22) PCT Filed: May 12, 2023
(86) PCT No.: PCT/EP2023/062721

§ 371 Date: Nov. 7, 2024

(30) Foreign Application Priority Data
May 13, 2022 (EP) 22173295.1
Sep. 20, 2022 (EP) 22196651.8

(51) Int. Cl. G01N 33/50; G01N 33/68

(52) U.S. Cl. CPC G01N 33/5005; G01N 33/6803

(57) ABSTRACT

The current invention relates to cell-cell interaction and in particular to cellular avidity. Provided are improved means and methods to study cell-cell interaction and characterizing cellular avidity. More in particular, the methods involve studying cell-cell interactions and applying forces in order to break cell-cell bonds in a controllable fashion. Analysing such interactions and breakage of cell bonds in time allows to assess cell-cell binding dynamics.

### FIELD OF THE INVENTION

The invention relates to means and methods for determining and measuring cellular avidity.

### BACKGROUND OF THE INVENTION

Despite tremendous clinical success of T cell therapy, including CAR-T cell therapy, for haematological malignancies, many obstacles still remain for the therapy in treatment of a wider range of cancers. Predicting success of T-cell therapy using in vitro data alone is still a challenge. Affinity of a binding molecule used for CAR generation towards the target antigen is frequently considered during the in vitro testing, but this does not define CAR-T cell activity. With the increasing complexity of constructing novel methodologies to further improve the clinical outcome of immunotherapies, screening methods to quickly identify the best lead candidates is becoming even more essential. To date, it has become evident that merely measuring the affinity between a CAR and its target will not accurately predict in vitro and in vivo outcomes. On the contrary, cellular avidity, the overall cellular binding strength, can provide a more complete and physiologically relevant measurement that reflects the bona fide interaction between T cells and target cells.

Cellular avidity measurements may better predict cellular responses in vitro, and drive better, more informed decisions at earlier stages for drug selection and potentially improve clinical outcomes. One of the main obstacles in the process of measuring avidity as being a critical parameter is the lack of fast, specific, and accurate tools to assess cellular avidity. The z-Movi® Cell Avidity Analyzer, a platform for measuring cell-cell binding strength facilitates a direct analysis of CAR-T cells either against surface immobilised antigens or a monolayer of target cells. Using this system, cell-cell interactions are perturbed using resonant sound waves generated by a piezoelectric element and a cell tracking system is employed to measure the required disruption force. This way, cellular avidity measurements can be done which provide for a cellular avidity score which allows to compare e.g. different candidate receptors.

The current inventors, in working with cellular avidity were now looking for means and methods to further improve cellular avidity measurements.

### SUMMARY OF THE INVENTION

The current inventors now provide for new and improved methods for assessing cellular avidity. The current inventors have compared different receptors and their effect on cellular avidity, as well as assessing conventional assays. The current inventors found that cellular avidity measurements, in particular when taking into account time, can provide valuable insights and allow for further differentiation between different receptors. Compared against traditional SPR data combined with cytotoxic and cytokine release under co-culture conditions, the cellular avidity methods in accordance with the invention can provide a more sensitive insight into e.g. CAR-T cell activity, also against cellular avidity measurements with a single set time, and was able to identify differences more readily than the standard in vitro assays. Cellular avidity measurements, taking into account different incubation times, have the potential of filling the knowledge gap currently present in the understanding of effector cell and target cell interactions and can complement current cellular avidity measurements utilizing a single set time and provide a new parameter highly useful in cellular avidity measurements and developing therapeutics involving effector cells and target cells, such as CAR-T cells or the like, for immunotherapy against cancer.

Accordingly, the current invention provides for a method of assessing cellular avidity of an effector cell carrying a receptor and target cells, comprising the steps of:
a) providing target cells;
b) providing effector cells carrying a receptor;
wherein the target cells or the effector cells carrying a receptor are attached to a surface;
c) contacting the effector cells carrying a receptor with the target cells to allow the effector cells carrying a receptor to interact with the target cells, wherein the interaction is for a first defined time;
d) applying a force, wherein the force is in a direction away from the attached cells, such that at least part of the cells bound to the cells attached to the surface move away therefrom;
e) detecting effector cells carrying a receptor that have remained bound with the target cells and attached to the surface after applying the force, and provide a cellular avidity score;
f) perform steps c), d) and e) with a second defined time.

Providing a cellular avidity score, e.g. as shown in the examples, includes i.a. determining the percentage of effector cells bound to target cells attached to a surface, relative to e.g. the effector cells initially provided. By determining the percentage bound effector cells obtained after different incubation times, and determining the difference, a further highly useful parameter can be provided. For example, in case of two different incubation times the difference in cellular avidity score can be calculated, e.g. CA (q)−CA (r), or the slope, e.g. CA(r)−CA(q)/q-r (wherein q and r are different incubation times). The cellular avidity score thus obtained may be referred to as Delta CA (e.g. %) or Slope CA (e.g. %/t), respectively. Such a parameter in addition to a CA at a defined time (e.g. at time r) may provide further insight into the capacity of the effector cell to bind a target cell, e.g. the so called on-rate. This way, different effector cells, or e.g. different cell engagers, may be compared and provide a parameter related to function that is highly useful in discovery and development. For example, effector cells or cell engagers equally capable of highly effectively binding target cells, but differing with regard to Delta CA or Slope CA.

### DESCRIPTION OF THE FIGURES

FIG. 1. Generation and functional testing of CAR-T cells. A) Expression levels of CAR were determined by staining the marker gene with PE-conjugated antibody and extrapolating the value using Quantibrite™ Beads (BD Biosciences™). B) 24 h cytotoxicity assay of 1:2 and 1:4 effector to target ratio, with from left to right UNT (untransduced cells), CAR1, CAR2 and CAR3. C) and D) show from left to right UNT, CAR1, CAR2 and CAR3, IFNγ and IL2 release from 24 h co-culture with target tumor cells.

FIG. 2. Cellular avidity measurement results. A) and B) show cellular avidity curves generated following 2 and 5 minute on-chip incubation time, i.e. the time for contacting and interaction, and during force application of a force ramp of up to 1000 pN. In both A) and B) the curves (from upper to lower curve) can be ascribed to CAR2, CAR1, CAR3 and UNT, respectively.

FIG. 3. Change in avidity of different CARs following different incubation times. The percentage of cells bound at 1000 pN was plotted for UNT, CAR1, CAR2, and CAR3 for the incubation times of 2 minutes and 5 minutes. At 2 minutes, the percentage of cells bound for CAR1 is lower (below 20%), and close to UNT, and CAR3 (about 0% and 10% respectively), as compared with CAR2 which has a much higher percentage of cells bound (above about 40%). Both CAR1 and CAR2 having a similar percentage of bound cells at 5 minutes, i.e. about 80%, whereas CAR3 and UNT have much lower percentages bound cells (about 20% and 10%, respectively). This results in a plot for UNT represented by the lower line, the second line from below representing the CAR3 plot, both having similar relatively flat slopes with low binding. The upper line represents the CAR2 plot, and the line below the upper line represents the CAR1 plot. The CAR1 plot shows a steep slope, whereas CAR2 has a less steep slope.

FIG. 4. In a cellular avidity measurement with cells attached to a surface (depicted as grey cells), cells (depicted as white cells) are interacted with the attached cells to bind therewith, e.g. utilizing target cells expressing an antigen and effector cells with a CAR against the antigen, as depicted in a). After a defined incubation, a force, Fₘ applied away from the attached cells. This results in cells moving away therefrom, either because they did not bind to the cells attached to the surface or because the binding strength was not strong enough, e.g. when aspecifically bound. Cells that remain were sufficiently strongly bound to the attached cells as depicted in b). Such a scenario is e.g. employed in the examples as described herein. Cells that formed a synapse may substantially remain (indicated with hashes). Alternatively, a cellular avidity measurement can be performed which does not require cells attached to a surface. In this scenario, e.g. target cells (depicted as white cells) are provided and effector cells (depicted as grey cells), and these are incubated for a defined period. After said incubation, cells are resuspended and a differential force, Fₙ, is applied. Such an applied force may be larger than typically used when cells are attached (Fₘ). This force may break cell-cell bonds, preferably aspecific cell-cell bonds, as these bonds may be less strong when compared with synapse bonds, thereby resulting e.g. substantially cell-cell bonds with a synapse. The cell suspension may be subsequently analysed with regard to singlets (either white or grey cells) and doublets (grey cell bound with white cell), which numbers may be used to provide a cellular avidity score. Of course, one may also combine applying a differential force (shown in d), after a force has been applied away from attached cells (on the scenario obtained as shown in b), or one may apply a differential force after incubation on attached cells (on the scenario as obtained in a)). Cellular avidity scores may be determined by determining the number of grey cells and white cells bound to each other (doublets) and/or by counting the number of grey cells bound to white cells, e.g. effector cells bound to target cells, that formed a synapse (doublets with hashes). Cellular avidity scores can be calculated taking into account the initial amount of cells provided and/or single cells obtained in the methods. For example, by calculating the ratio of the number of effector cells that remained bound to target cells after exerting the force to the number of effector cells initially provided.

FIG. 5. Cellular avidity measurement results. FIG. 5 shows cellular avidity curves generated following 2.5, 5 and 10 minute on-chip incubation times, i.e. the time for contacting and interaction, and during force application of a force ramp of up to 1000 pN over 2.5 minutes. The upper curve can be ascribed to CAR1 transduced cells, while the lower curve can be ascribed to untransduced cells.

### DETAILED DESCRIPTION OF THE INVENTION

Hence, as said, the current inventors working with means and methods for determining cellular avidity now provide for a further method highly useful for assessing cellular avidity, which involves determining cellular avidity scores with different incubation times. In one embodiment, the invention provides for a method for assessing cellular avidity of an effector cell carrying a receptor and target cells, comprising the steps of:
a) providing target cells;
b) providing effector cells carrying a receptor;
wherein the target cells or the effector cells carrying a receptor are attached to a surface;
c) contacting the effector cells carrying a receptor with the target cells to allow the effector cells carrying a receptor to interact with the target cells, wherein the interaction is for a first defined time;
d) applying a force, wherein the force is in a direction away from the attached cells, such that at least part of the cells bound to the cells attached to the surface move away therefrom;
e) detecting effector cells carrying a receptor that have remained bound with the target cells and attached to the surface after applying the force, and provide a cellular avidity score;
f) perform steps c), d) and e) with a second defined time for step c).

Target and effector cells carrying a receptor are provided. Target cells in accordance with the invention are the cells on which the effector cells are to exert an effect, e.g. bind therewith and trigger an immune reaction thereto. Target cells include cancer cells presenting an antigen. An antigen may be presented by MHC, i.e. HLA in humans, which are specialized receptors that present peptides e.g. derived from digested proteins expressed by the cell (e.g. usually 8-11 amino acids in length for MHCI). An antigen may also be a protein or other biomolecule that is presented on the surface of a cell, e.g. epidermal growth factor receptors or checkpoint proteins, which in the case of cancer cells are overexpressed therewith providing a differentiating feature. Target cells may also include cells expressing auto-antigens, e.g. known to be involved in autoimmunity diseases or cells infected with a pathogen, e.g. a virus. In accordance with the invention, effector cells carrying a receptor include effector cells of the immune system that can exert an effect, via the receptor. For example, a T cell carrying a T cell receptor can bind an antigen on a cancer cell, upon which it can e.g. exert a cytotoxic effect and kill the target cell. Effector cells can be derived from nature, e.g. obtained from a host, and can also include genetically modified cells, wherein e.g. a receptor in particular useful is provided to an effector cell.

Subsequently, either the target cells or the effector cells are attached to a surface. It is understood that surfaces for attaching cells may be any surface suitable for attaching cells. Suitable surfaces for attaching cells include plastic or glass surfaces. These surfaces may be coated e.g. with a protein to attach cells to the surface, such as poly-L-lysine (PLL) or the like. The attachment of the cells to the surface is such that the strength of the binding to the surface is sufficient to have the cells remaining attached when applying a suitable force.

The effector cell carrying the receptor is capable of binding target cells, or is studied for its capability of binding target cells. It is understood this capacity of binding target cells can include a specific interaction that can induce synapse formation, i.e. the effector cell is to bind to a target cell and exert an effect thereon, e.g. induce target cell killing.

It is understood that a synapse is a specialized structure that forms when the plasma membranes of two cells come into close proximity to transmit signals. Cells of the immune system form synapses that are essential for cell activation and function. Lymphocytes such as T cells and natural killer (NK) cells form synapses that can be referred to as immunological synapses. Such a synapse typically forms between immune effector cells and target cells, e.g. cells presenting an antigen. A non-limiting example is e.g. a T cell or a CAR-T cell and a cancer cell, such as used in the examples as described herein. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the next step, effector cells and the target cells are contacted with each other to allow the cells to interact. This step is such that the effector cells will have sufficient time to interact with target cells and can form a bond, including synapses. It is understood that a cell-cell interaction may not always result in a cell-cell bond, which can be a synapse or aspecific bond, the contacting step is such that cell-cell bonds can be formed and appropriate conditions therefore are selected. It is understood that because the conditions are selected such that a synapse can be formed, this necessarily implies that aspecific cell-cell bonds are allowed to be formed at the same time. This contacting step is to be performed for a defined first time.

Once the effector cells have had contact with the target cells for a defined time, and thus have had the opportunity, if possible, to form a synapse, in a subsequent step a force is applied away from the cells attached to the surface, such that at least part of the cells bound to the cells attached to the surface, and unbound cells as well, move away from the cells attached to the surface. This way, target cells are obtained with effector cells bound thereto, which are attached to the surface, wherein the exerted force was not sufficient to break cell-cell bonds. Of course, cells that are attached to the surface, to which no subsequent cells are bound, remain as well.

As said, the force is applied in a direction away from the attached cells. It is understood that in this step, the force applied may be perpendicular (in the direction of z-axis) to the surface (x,y) to which cells are attached, for example when a centrifugal force or acoustic force is applied. The force may also be lateral (in the direction of the x-axis or y-axis relative to the surface), for example when a shear force is applied. In any case, the force is applied and is controlled such that a defined force is exerted on the cells that interacted with the attached cells. It is understood that the force that is exerted on the cells interacting with attached cells is to be substantially equal, such can be achieved e.g. when using a flat surface. Other suitable surface shapes may be used (e.g. a tube with exerted concentrical force or laminar flow force in the direction of the length of the tube), as long as the force exerted can be substantially equal at a defined surface area to which cells are attached, such a surface shape may be contemplated.

The cells that are attached are preferably attached to a glass or plastic surface, preferably a surface in a chip, which allows for detection of cells e.g. via microscopy or other means. The applied force required to move a cell away from an attached cell preferably can be detected, e.g. via microscopy or other means, to which may be referred to as a cell detachment event or cells moving away. This way, cells moving away can be monitored and counted. It may be advantageous and convenient to use microscopy, with which bound cells can be identified and quantified and cells moving away can be likewise monitored and quantified, also allowing e.g. to detect markers. For example, the z-Movi® device as available from Lumicks which applies an acoustic force may be well equipped to do so. Likewise, similar devices may be provided with microscopy or other means to quantify cells, detect markers and bound cells and/or cells moving away, and also utilizing e.g. shear force or centrifugal forces instead of acoustic force.

In any case, effector cells carrying a receptor that have remained bound with the target cells, and are attached to the surface after applying the force, are detected. Furthermore, a cellular avidity score is provided. It is understood that a cellular avidity score can be determined based on the number of cells that have moved away and/or remain bound after the force has been exerted. It is understood that the cellular avidity score highly preferably is to be determined based on the cells that were not attached to the surface. For example, when the target cells are attached to the surface, and the effector cells provided, the cellular avidity score can be determined utilizing the determined number of effector cells that have moved away and/or remain bound after the force has been exerted.

A cellular avidity score can for example be calculated by determining the ratio of the number of effector cells that remained bound with the target cells to the number of effector cells that was initially provided or to the number of effector cells that moved away. As is understood herein, the ratios determined may be based on absolute numbers, or may be calculated based on numbers determined of fractions thereof. For example, one may determine of a defined surface with cells attached, the number of subsequent cells that interact therewith and detect each cell that moves away from the attached cells and determine the number of cells at the defined surface that remain after the force has been exerted. One may represent calculated ratios as a number or as a percentage, such as shown e.g. in the example section herein. In any case, determining the number of cells that have moved away and/or remain bound after the force has been exerted allows one to provide for a cellular avidity score that is a highly useful measure which is indicative of how well the effector cell carrying the receptor can bind with a target cell. The more cells that remain bound and/or the less cells that move away, the higher the cellular avidity is, which is reflected in a calculated cellular avidity score which allows for comparing e.g. different effector cells.

Subsequently, the contacting step, in which effector cells are interacted with the target cells, the step of applying the force, and the detection step, which includes providing a cellular avidity score, are repeated, now with a second defined time for the contacting step. This second defined time being different from the first time.

It may be preferred to carry out the method such as described in the examples, i.e. having the target cells attached to a surface. Hence, in another embodiment, a method is provided of assessing cellular avidity of an effector cell carrying a receptor and target cells, comprising the steps of:
a) providing target cells attached to a surface;
b) providing effector cells carrying a receptor;
c) contacting effector cells carrying a receptor with the target cells to allow the effector cells carrying a receptor to interact with the target cells, wherein the interaction is for a first defined time;
d) applying a force on the effector cells carrying a receptor, wherein the force is in a direction away from the target cells;
e) determine effector cells carrying a receptor that have detached and/or remain bound in step d) and provide a cellular avidity score;
f) perform steps c), d) and e) with a second defined time for step c).

As said, the current inventors working with means and methods for determining cellular avidity now provided for a further method highly useful for assessing cellular avidity between different cells, which involves determining cellular avidity scores with different incubation times. It is understood that this method may not be restricted to effector cells carrying a receptor, such as e.g. T-cells, and target cells, such as e.g. cancer cells. It is understood that the means and methods of the invention may also be employed to study cell-cell binding dynamics. Hence, in another embodiment, the invention provides for a method for assessing cellular avidity of a first cell and a second cell, comprising the steps of:
a) providing first cells;
b) providing second cells;
wherein the first or the second cells are attached to a surface;
c) contacting the first cells with the second cells to allow the first cells to interact with the second cells, wherein the interaction is for a first defined time;
d) applying a force, wherein the force is in a direction away from the attached cells, such that at least part of the cells bound to the cells attached to the surface move away therefrom;
e) detecting first cells that have remained bound with the second cells and attached to the surface after applying the force, and provide a cellular avidity score for the first and second cells;
f) perform steps c), d) and e) with a second defined time for step c).

It is understood that the method in accordance with the invention is to be performed with at least two different defined times, i.e. a first and a second defined time. This obviously does not exclude the possibility to perform the method with further defined contacting (or incubation or interaction) times. Hence, in another embodiment, the contacting step, in which effector cells are interacted with the target cells, the step of applying the force, and the detection step, which includes providing a cellular avidity score, are repeated, with a third, and optionally more, defined time(s) for the contacting step.

With regard to the two or more defined times, these are preferably selected from the range of 1 second to 4 hours, from 1 second to 3 hours, from 1 second to 1 hour, from 1 second to 40 minutes, from 1 second to 30 minutes, from 1 second to 25 minutes, from 1 second to 20 minutes, from 1 second to 15 minutes, from 1 second to 10 minutes. With regard to the two or more defined times, these can also preferably be selected from the range of from 10 seconds to 30 minutes, from 30 seconds to 30 minutes, from 1 minute to 30 minutes, from 10 seconds to 10 minutes, from 30 seconds to 10 minutes, or from 1 minute to 30 minutes. The two or more defined times may be selected from 1 second, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, and 15 minutes. The two or more defined times may be selected from 1 minute, 2 minutes, 3 minutes, 4 minutes, 5 minutes, 6 minutes, 7 minutes, 8 minutes, 9 minutes, and 10 minutes.

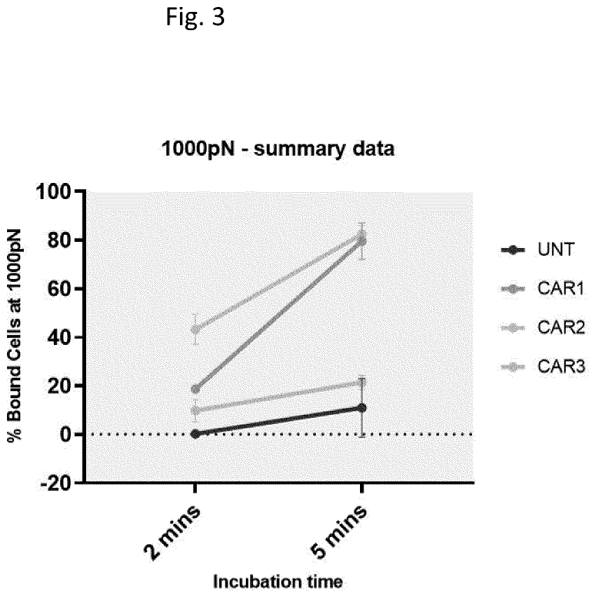

As shown in the example section, the determined cellular avidity scores determined for the defined times can subsequently be plotted, e.g. plotting time at an x-axis and cellular avidity score at the y-axis. For example, as shown in the example section, and as shown in FIG. 3, providing such a plot may be useful, e.g. as it may be indicative of a further differentiating parameter. For two defined times plotted, the slope of the line connecting the two plotted measurements can be determined. Alternatively, one can also determine the difference in cellular avidity score between two time points. Hence, it is understood that it is not necessary to determine a slope, but this is optional. When more than two time points are plotted, the maximal slope may be determined between two time points, or alternatively, the maximal difference observed between two defined time points, or the ratio. To such a slope may be referred to as a Slope CA, or to such a difference may be referred to as a Delta CA or to a ratio may be referred to as Ratio CA which may be calculated e.g. with a formula such as Delta CA=CA (q)−CA (r), Slope CA=(CA(r)−CA(q))/q-r, or Ratio CA=CA (q)/CA (r) (wherein q and r are different incubation times). Having multiple time points may be advantageous, as it may allow to plot a curve, which can provide further insights as well. Plotting a curve may e.g. allow to determine the maximal slope that can be obtained between two time points. Furthermore, it may also be determined if a plateau is reached for the cellular avidity score determined, e.g. from which incubation time onwards the cellular avidity score that is determined may no longer substantially increase further. Hence, in a further embodiment, wherein when multiple cellular avidity scores are determined and plotted against time, the plateau of the plot is determined, e.g. the cellular avidity score of the plateau and/or from which incubation time onwards this plateau is substantially reached.

It is understood that the cellular avidity scores determined at the first and second defines times, and optionally further defined times, highly preferably is determined with a defined force. Such a defined force most preferably being the same for all measurements. It is also understood that in accordance with the invention, incubation, i.e. contacting and interacting are defined as being separate from the step of applying the force. It is understood that of course while applying a force, cells may remain in contact and interact, depending on the amount of force exerted, e.g. in case of a force ramp with perhaps an initial low amount of force. In any case, it is highly preferred to apply the same defined force (which includes force ramps) and varying the contacting/interaction (i.e. incubation) time. One may of course also contemplate to apply a (minor) force for different defined periods as part of e.g. a force ramp, which essentially involves the same principle of applying different incubation times.

Accordingly, as described above, cellular avidity scores as determined at different time points allow to provide further insights, e.g. by providing a further parameter. This may be highly useful for example when comparing effector cells which are provided with different receptors. Such a comparison includes a comparison of cellular avidity scores, and can include comparing e.g. determined slopes as well. Of course, highly preferably, the same target cells or types of target cells are used to allow such a comparison. It is understood that cellular avidity scores thus obtained may be referred to as cellular avidity scores of the receptors or of the effectors cells carrying the receptor, as the target cells are the same. See e.g. table 1 as listed in the example section wherein cellular avidity scores are listed for different incubation times, and Delta CA, Slope CA, and Ratio CA, were determined as well. Comparing the results obtained may include a comparison based on determined slope and/or plateau reached. As shown in the examples, as only two time points were used, it cannot be established whether a plateau was reached, in such a scenario, one may select the maximum CA determined. As shown in the example section, CAR1 and CAR2 had a similar maximum cellular avidity score, with the Delta or Slope CA being higher for CAR1 than for CAR2. In any case, effector cells e.g. being provided with different receptors can be ranked based on slope and/or plateau, and/or observed maximum cellular avidity score. It is also understood that different effector cells may be provided with different receptors and/or the same receptor which may also be ranked based on slope and/or plateau. Effector cells with a receptor having a high slope and a high plateau may be provided with a high rank, e.g. because the high plateau can indicate efficient target cell binding and e.g. subsequent killing, while having a high slope may be informative of the on-rate, and a high slope may be indicative of a slow(er) on-rate. In any case, candidate receptors may be ranked, and based on the ranking, selection of candidate receptors can be made for further experiments, i.e. to allow for candidate receptors differing with regard to e.g. Delta CA or Slope CA, which may include features related to maximum cellular avidity observed, or plateaus reached as well.

As said, the force applied with the two or more incubation times selected, preferably is the same with the two or more incubation times selected. In another embodiment, the applied force is a force ramp, preferably a linear force ramp. It is understood that the force that is to be applied can be a constant force applied for a defined period. The forces applied may be in various forms as a function of time. Preferably however, the applied force is an increasing force, that is, after the contacting step, an increasing force is applied for a defined period until a defined end force is reached. Increasing the force is preferably done as a linear force ramp, but other ways to increase the force over time may also be used (e.g. exponential loading where the force is doubled over a certain small time period and keeps doubling until an end force is reached). For example, as shown in the example section, in about 150 seconds, a defined end force is reached of 1000 pN. An increasing force may be referred to as a force ramp, which may preferably be a linear force ramp, but other ways of increasing the force may be contemplated. The applied force is selected such that the cells that are attached to surface remain attached. Moreover, the applied force selected may also depend on the amount of background observed, with higher forces exerted generally allowing for less background binding to be observed. A suitable applied force may be selected from the range of 1-3000 pN. More preferably, the force exerted, e.g. at the end of a force ramp, is in the range of 200 pN-3000 pN.

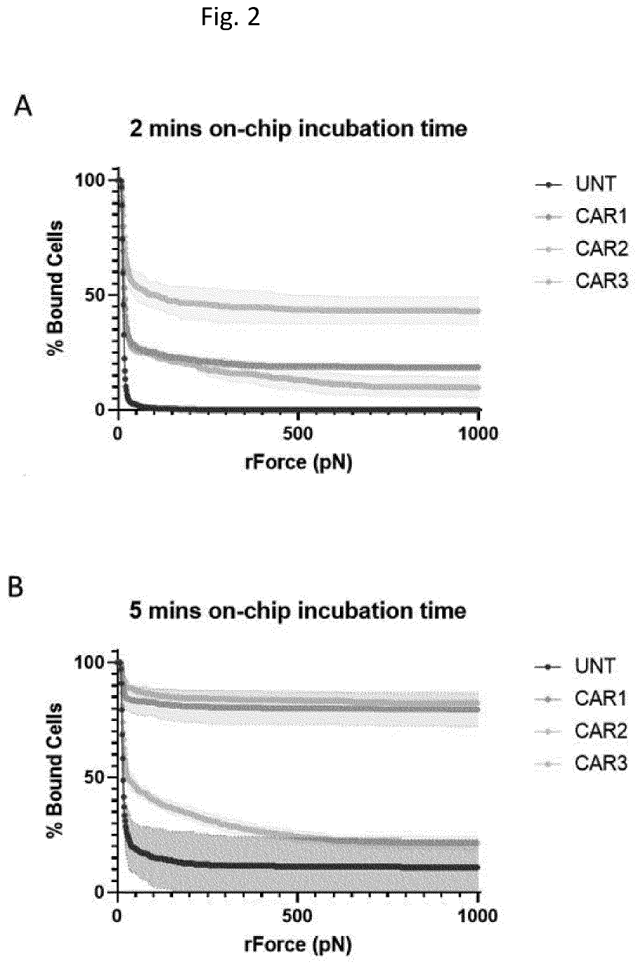

As said, detached cells can be collected in a fraction. It may also be opted to collect cells in different fractions, e.g. each fraction representing a defined period in which the force (ramp) was applied. By collecting different detached fractions, one may e.g. construct a cellular avidity curve, i.e. plotting determined fractions at the y-axis, while plotting increasing applied forces at the x-axis (see e.g. FIG. 2).

Any suitable force application method may be contemplated in accordance with the invention. Increasing the force can be well controlled with acceleration-based methods of applying force such as centrifugation, with shear flow and with acoustic force, which are all suitable means to be used in the methods in accordance with the invention but any other means of controllably causing a force on the cells attached to the surface-attached cells, thereby forcing them away from the surface-attached cells or the functionalized wall surface, may be contemplated. Accordingly, in a further embodiment, the force that is applied is selected from an acoustic force, a shear flow force or a centrifugal force.

It may be advantageous to apply shear flow force and/or centrifugal force in scenarios wherein a large number of cells are to be tested. Acoustic force, though very well suitable in the means and methods of the invention, allows for the processing of about 100-10,000 cells, e.g. utilizing a device like the z-Movi© as available from Lumicks. This is because the surface area at which the acoustic force may be well controlled can be limited. However, using centrifugal force and/or shear flow force, the forces exerted on the cells that attach to the target cell layer can be more easily controlled over much larger areas allowing for the sorting of millions to billions of cells.

With regard to the cellular avidity and scores, it is understood that this is indicative of the strength of binding of cells of interest, i.e. cells carrying a receptor (or control cells thereof) to the target cells. It is understood that where we refer to specific forces applied to cells this may refer to average forces, e.g. such forces may not be fully homogeneous, for example over the contact surface as may be the case with acoustic forces and shear flow forces (see e.g. Nguyen, A., Brandt, M., Muenker, T. M., & Betz, T. (2021). Lab on a Chip, 21(10), 1929-1947) for a description of force inhomogeneities in acoustic force application).

Also, for shear flow forces the forces may not be fully homogeneous, for example since the flow speed near the side walls of a flow channel (e.g. with a rectangular cross section) may be lower than in the centre of the flow cell (due to the no-slip boundary condition). By choosing a cross section with a high aspect ratio (low and wide) these flow effects may be minimized such that only a few percent of the cells experience a substantially smaller force than the cells in the centre of the flow cell. Other methods to mitigate such effects and to specifically select cells that have experienced similar forces may include using flow cell geometries with multiple fluid inlets and/or outlets such that the properties of laminar flow can be used to ensure cells of interest only land in regions of homogeneous force and/or are only selected from regions of homogeneous force. In one example, by using three channel inlets side by side one can use the side channels as sheath flow channels to focus cells of interest inserted into the central channel inlet towards the centre of the interaction region where the acoustic and/or shear force may be substantially homogeneous. The sheath flow fluid may be the same buffer fluid as is used for the sample cells but then free of sample cells. By increasing the flow speed through the sheath flow channels the cells are more focused and confined to the centre of the channel while by reducing the sheath flow speed the cells are allowed to spread out more. Similarly, on the collection side, flows in three side-by-side collection channels may be controlled to possibly discard cells flowing close to the channel boundaries and only collecting cells from the centre of the channel. By controlling the relative flow speeds of such side channels and the centre channel asymmetrically the location of the effective interaction region of the sorting device can be further controlled and cells that have underwent defined forces can be selected and/or detected.

Further means to enable collection of cells from a specific interaction region (and therefore collection of cells that experienced a defined force) include means and methods wherein cells of interest may be provided with a photoactivatable label which may be subsequently activated by illumination with light of a suitable wavelength only in a well-defined interaction region of the device (e.g. near the centre of a flow channel or in a centre region under an (acoustic) force transducer) to photoactivate and/or switch the dye. Subsequently, the cells can be sorted for example using fluorescence activated cell sorting (FACS) and only those cells which are activated are further used according to the methods described herein thereby obtaining the cells on which defined forces have been exerted.

For centrifugal forces, it is easier to ensure that the force applied is homogeneous across the whole interaction region, since such a force does not depend strongly on a location on a surface with respect to a force transducer and/or the wall of a flow channel or sample holder.

Accordingly, in connection to the subject matter disclosed herein, means and methods exist which allow one to exert forces on cells attached to a surface and collect the cells, detached and/or attached cells, on which defined forces have been exerted.

It is understood that with regard to the force exerted, the exact forces experienced by cells may also depend on cell size and or other cell properties such as density and compressibility (the force may be a nominal force and not the true force experienced by the cells, e.g. it may be hard to precisely predict the average cell size, density, compressibility, etc. of the cells and the force may have been calculated based on theory alone or may have been calibrated using test particles with specific preferably known properties (see e.g. Kamsma, D., Creyghton, R., Sitters, G., Wuite, G. J. L., & Peterman, E. J. G. (2016). Tuning the Music: Acoustic Force Spectroscopy (AFS) 2.0. Methods, 105, 26-33)). The force may be such a calculated or calibrated force expressed with units of N (e.g. pN), but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014). Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices such devices are suitable for the means and methods in accordance with the invention.

In another embodiment, the target cells are attached to a glass or a plastic surface, preferably a glass surface in a chip or the like (such as described i.a. in Fernández de Larrea, C. et al. (2020). Blood Cancer Discovery, 1(2), 146-154) with target cells attached to its surface. Such a chip is also described in U.S. Pat. No. 10,941,437 B2 and WO 2018/083193. The target cells that are attached to the surface, preferably are attached as a monolayer. The monolayer preferably is at high confluency. The subsequent cells that are to interact with the target cells are preferably provided in a relatively low cell density as compared with the target cells, such that substantially all cells can interact with a target cells (there are more target cells available for the total number of cells comprised in the effector cells). Such provides for advantageous controllable conditions when applying the force on the cells, e.g. on the effector cells.

Further embodiments include methods being performed in the presence of agent(s). For example, one or more agents capable of modulating the cellular avidity between an effector cell with a receptor and a target cell may be included in the steps of contacting effector cells with target cells and subsequently applying a force away from the target cells. Modulating is understood to either enhance or reduce cellular avidity. This way, for example, in case an agent is present that is known to modulate a desired interaction between a cell with a receptor and a target cell, receptor/target interactions may be selected that are not affected by such agents, or, conversely, are aided by such agents. Hence, in another embodiment, an agent capable of modulating the interaction between the effector cells with a receptor and the target cell is included at least in the contacting step and when applying a force. For example, an agent could be used that reduces aspecific binding between a cell with a receptor and target cells.

In another embodiment a cell engager may be provided. Cell engagers include antibodies, or the like, which are capable of binding to a target cell and an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, e.g. synapse formation, of induced by a cell engager between a target cell and an effector cell. Hence, accordingly, in the means and methods in accordance with the invention, a cell engager may be provided in addition, and e.g. the cellular avidity score is determined, induced by said cell engager between an effector cell and a target cell, said cell engager having a binding region capable of binding the effector cell and a binding region capable of binding the target cell. It is understood that the contacting step in the methods of the invention can thus be performed in the presence of the cell engager to allow the cells to interact, i.e. effector cells and target cells, and form a bond, e.g. a synapse, via the cell engager.

Accordingly, in one embodiment, in methods in accordance with the invention, a cell engager is provided capable of binding the effector cell and the target cell and inducing synapse formation, and the cell engager is included in the contacting/interacting step. Furthermore, such methods are highly useful for screening cell engagers, e.g. by identifying cell engagers that are particular capable of inducing a synapse.

It is understood that a synapse is a specialized structure that forms when the plasma membranes of two cells come into close proximity to transmit signals. Cells of the immune system form synapses that are essential for cell activation and function. Lymphocytes such as T cells, B cells and natural killer (NK) cells form synapses that can be referred to as immunological synapses. Such a synapse typically forms between effector cells and target cells, e.g. cells presenting an antigen. A non-limiting example is e.g. a T cell or a CAR-T cell and a cancer cell. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the case of a T cell, a synapse can be formed between the lymphocyte and antigen-presenting cells (APCs) during the recognition of the peptide antigen-major histocompatibility complex (pMHC) ligand by the T-cell antigen receptor (TCR). The TCR and pMHC are both membrane-bound so the TCR will only be triggered by its ligand at the interface between T cells and APCs. A synapse can be observed at the T cell—APC interface as concentric rings by confocal microscopy, often referred to as “bull's eye” (Huppa, J. B., & Davis, M. M. (2003). T-cell-antigen recognition and the immunological synapse. Nature Reviews Immunology, 3(12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T-cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2), CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2), CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR-T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g., CD28, 41BB, ICOS) incorporated in the cytoplasmic tail of the CAR—To enhance signalling further. Further generations also incorporated in their design an inducible release of transgenic immune modifiers, such as IL-12, to shape the tumor environment by augmenting e.g. T-cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized pattern. Multiple CAR micro clusters form and signalling molecules, which are dispersed in the centre of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved in T cell activation and/or synapse formation. Sequencing based methods may also be used to identify activated T cells and thereby associate mRNA levels with the formation of stable synapses. T cell activation leads to changes in mRNA stability and expression. E.g. increases in expression of cytokine or secretory transcripts (IL2, IFNγ, granzyme, perforin) and proliferation pathways and either bulk or single-cell RNA sequencing may be used to detect these changes and correlate these to the number or synapse formed.

In any case, in the cells obtained, which can be either in the form of target cells bound to effector cells or separated cells, e.g. with trypsin, the marker associated with synapse formation can be determined. Thus, in a further embodiment, the marker associated with synapse formation is determined in either the target cell or the effector cell. In another embodiment, in the methods in accordance with the invention one or more markers associated with synapse formation are determined and the one or more markers are determined in the effector cells and/or in the target cells. When cells are labelled, either in a singlet state or doublet state, cells may be highly advantageously be sorted and collected and subsequently analysed.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse in which, as described above but not necessarily limited thereto, markers associated with synapse formation are associated.

In a further embodiment the marker for synapse formation is calcium signalling, which can be detected with fluorescent calcium indicators, such as Fura2 AM (available from Invitrogen, item nr. F1221), which marker is suitable for detection in effector cells and in live cells. Other suitable dyes to detect calcium signalling can be selected from the group of Fura Red AM, Indo-1, penta potassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, penta potassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used a calcium signalling indicators, such as the Invitrogen FluoVolt™ Membrane Potential Kit (Catalog number: F10488). Depolarization of the synapse forming cells by using a slow-response potential-sensitive probe such as Invitrogen DiSBAC2(3) (Bis-(1,3-Diethylthiobarbituric Acid)Trimethine Oxonol), Catalog number: B413. Hence, in another embodiment the marker for synapse formation is detected utilizing membrane potential dyes.

In another embodiment, the marker for synapse formation is cytoskeleton rearrangement, which can be detected in effector cells with live or fixed cells, using cell staining, e.g. F-actin can be detected with Phalloidin conjugates or CellMask™ (Invitrogen, item nr A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is detected with a stain selected from the group consisting of Phalloidin conjugates, CellMaskTMh SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation effector cells will stop moving and remain into contact with the target cell with which it forms a synapse. Cell motility can be detected by membrane staining of effector cells and imaging, or using brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cell for which the marker associated with synapse formation was detected can easily be tracked.

As described above, in accordance with the invention, markers associated with synapse formation can be determined, e.g. via utilizing labels or the like. However, it may be highly advantageous to sequence obtained cells and identify synapse formation by sequencing. It is understood that sequencing comprises nucleotide sequencing, e.g. sequencing of DNA and/or RNA as expressed in cells. Means and methods are widely known in the art to sequence DNA and/or RNA as expressed in the cell. Hence in another embodiment, in the methods in accordance with the invention the markers are determined with sequencing. This is in particular highly useful for such markers which are up- or downregulated in target cells and/or effector cells in forming a synapse or having a synapse. Suitable markers for T cell activation and synapse formation are transcripts linked to interferon expression, proliferation, and cytokine expression and include: Interferon pathway upregulation: CD4, IFIT3, IFIT2, STAT1, MX1, IRF7, ISG15, IFITM3, OAS2, JAK2, SOCS1, TRIM21; proliferation: LIF, IL2, CENPV, NME1, FABP5, ORC6, GOS2, GCK; cytokine expression: CCL3, IFNG, CCL4, XCL1, XCL2, CSF2, IL10, HOPX, TIM3, LAG3, PRF1, TNFRSF9, NKG7, IL26. Sequencing may also be used to identify non-synapse forming cells by detecting molecular signatures linked to resting cell states: FOXP3, CTLA4, MTNFRSF4, IRF4, BATF, TNFRSF18, TOX2, PRDMI, LEF1, ATM, SELL, KLF2, ITGA6, IL7R, CD52, S100A4, TGFB3, AQP3, NLRP3, KLF2, ITGB7.

It may be also advantageous to determine the molecular state of the target cells as this can give an indication of the cell killing potency of the effector cells. In the case of paired analysis were effector-target-cell doublets are recovered this enables direct linking of effector cell phenotype with their killing capabilities. Next to staining methods for apoptosis commonly used in the field, sequencing or qPCR can be used to detect transcripts linked to apoptosis or cell survival in the target cell, markers include: Bcl-2 family (BCL-xL) caspases 3, caspases 7, cleaved PARP, bax, bad, bak, bid, puma noxa, bcl-2, bcl-xl, mcl-1, p53, and cytochrome c, Smac/Diablo, survivn, Mcl-1, RNA Y1.

As it is understood that changes in gene expression may take some time and are not necessarily immediately detectable, one may allow after the step of exerting the force, the cells to remain bound to the target cells for some time and have these remain attached to the surface as well. Alternatively, one may also separate e.g. doublets that remained after exerting the force and subsequently e.g. sort doublets in single wells, and allow these to remain for some time. A suitable time to allow for expression of markers associated with synapse formation may be from 1 hour to 24 hours.

This is highly advantageous as sequencing methods allow for single cell sequencing, which also allows for determining the sequences of receptors expressed by the cell as well. Hence, in a further embodiment, different receptors are determined and identified via sequencing. In yet another embodiment, sequencing comprises single cell sequencing. In still another embodiment sequencing comprises sequencing the expressed genome. Sequencing the expressed genome is highly useful as it allows to determine up- and downregulated gene expression. Nevertheless, sequencing genomic DNA may be useful as it may also provide useful information e.g. epigenetic markers associated with in vivo potency and durable response.

With regard to the cellular avidity score as determined herein in accordance with the invention, it is understood that this takes into account the number of cells that have remained bound to each other after applying the force. Advantageously, this may also take into account the number of cells that remained bound to each other having one or more markers associated with synapse formation. Such a cellular avidity score which takes into account target cells and effector cells having a marker associated synapse formation may provide for a more accurate cellular avidity score which is more reflective of the function of e.g. effector cells, i.e. forming synapses with target cells. Hence, such as cellular avidity score may also be referred to as a functional cellular avidity score or a synaptic cellular avidity score, and it is understood that where herein cellular avidity scores are determined, this may also include cellular avidity scores taking into account synapse markers.

It was observed that when cells that remained bound and attached after a cellular avidity measurement were resuspended utilizing i.a. repeated pipetting and thus exerting a significant force, cells attached to the surface were detached and moreover, a portion of the cells that were bound to the attached cells became unbound. Hence, this implied that with such a process step a differential force can be applied on bound cells that can exceed the maximum force that is applied away from cells attached to a surface. This way, further cell-cell bonds that may be aspecific cell-cell bonds may thus be broken therewith providing substantially effector cells bound with target cells (and optionally a cell engager) that formed a synapse can be retained. Hence, in a further embodiment, after the force has been applied away from the attached cells, subsequently, the cells are resuspended and a differential force is applied such that substantially cells that are bound to each other via a synapse remain bound to each other and substantially cells that have formed aspecific bonds are unbound, i.e. have their cell-cell bonds broken. It is also understood that instead of applying the force away, the differential force may be applied instead, and that by applying a differential force, it may no longer be required to have cells attached to a surface, i.e. immobilized. Furthermore, it is also understood that after the differential force has been applied, cells are highly preferably separated and/or sorted in order to avoid further interaction between cells to avoid establishing additional cell-cell bonds. Hence, highly preferably, cells are collected and sorted and/or analysed after the differential force has been applied.

As is clear from the above, the type of force that is to be applied in accordance with the invention is a force capable of breaking cell-cell bonds, i.e. the force exerted causes cells bound to each other move away from each other to such an extent that a cell-cell bond may break or rupture. A differential force means that the force on one cell differs from the force on the other cell with regard to direction of the force and/or the magnitude of the force, resulting in a net force allowing to break cell-cell bonds if the differential force exceeds the binding force.

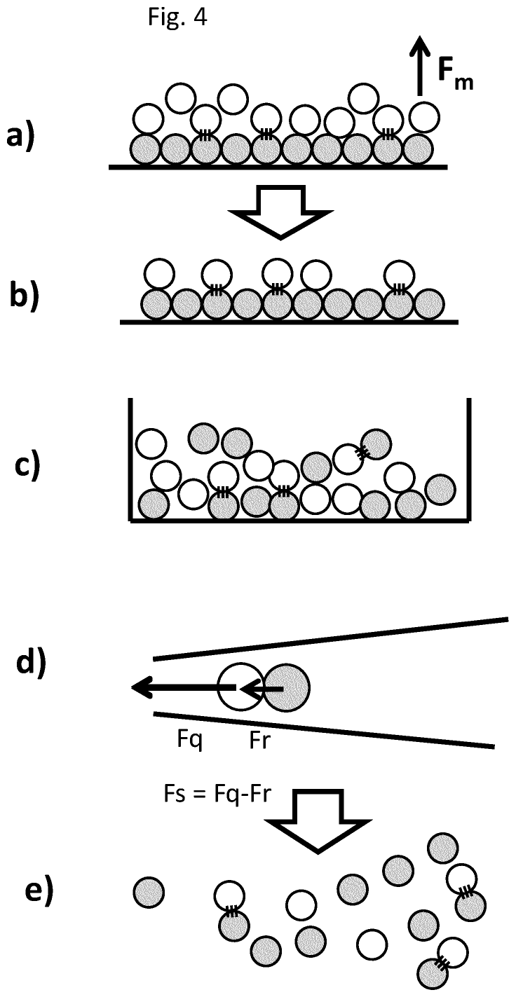

For example, when a target cell bound to an effector cell, a doublet, is forced through a nozzle, the closer to the throat of the nozzle the faster the flow. This means that the first cell to enter the nozzle is subjected to a stronger acceleration than the cell lagging behind and the cells experience a differential force resulting in a net force which can result in cell-cell bond rupture, provided the force is large enough (see e.g. FIG. 4, in particular 4d). A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J. Histochem. Cytochem. 1979, 27 (1), 635-641). Also, by properly tuning the forces in a flow-cytometer normally used to measure cell deformations, such as e.g. described in Otto, et al. Nat. Methods 12, 199-202 (2015) suitable forces can be applied. Tuning can be achieved e.g. by changing the nozzle size or geometry and/or the flow speeds used. Other suitable devices known in the art may include for a vortex mixer, with which shear forces may be suitably applied as well. Accordingly, in one embodiment, the force applied involves a shear force.

In another embodiment the force applied is an ultrasonic force. It is understood, as outline above, that such ultrasonic forces are not forces such as applied e.g. in a device as available from Lumicks, wherein the force is away from attached cells (e.g. such as in the LUMICKS z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter 11.2.8, page 576; and Moore et al., Experimental Cell Research, Volume 65, Issue 1, 1971, Pages 228-232).

In any case, suitable applied forces which are known in the art include e.g. a force in the range of 50 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 50 pN, or at least 100 pN, or at least least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. For example, a suitable amount of force that can be exerted between cells (e.g. such as in the z-Movi© device) can be selected to be in the range of 200 pN-3000 pN. Of course, these force ranges are known to be useful with cells attached to a surface, and the maximum force that may be selected may exceed 3000 pN as it is not required to have the cells attached to a surface in accordance with the invention.

Accordingly, it is understood that in these embodiments, the differential force to be applied does not require either of the target cells or effector cells to be attached, and the differential force is a force selected from the range of 50 pN-10 nN. In another embodiment, in methods in accordance with the invention wherein the force that is applied is a differential force, neither the target cells nor the effector cells require to be attached to a surface, and the differential force is applied in the range of 50 pN-10 nN, thereby providing cells substantially comprised of target cells, effector cells, and effector cells bound with target cells via a synapse.

Without being bound by theory, as is understood the range of force that may break an aspecific cell-cell bond versus a specific cell-cell bond that forms a synapse differs. This difference can be to such an extent that the ranges of the required forces do not overlap. It is understood that some overlap may occur. Hence the force that is selected, as outline above, may allow for aspecific cell-cell bonds remaining and some specific cell-cell bonds that formed a synapse to break. In case there is substantially no overlap, a differential force can be selected, as outline above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed a synapse. As also outlined herein, the portion of cell-cell bonds that remains after exerting a force and has a synapse, can be determined by determining the presence or absence of a marker associated with synapse formation.

In one embodiment, after the step of applying a force in the methods of the invention, the cells are resuspended and a subsequent differential force is applied such that formed aspecific cell-cell bonds are broken. In another embodiment, in methods of the invention wherein either the target cells or effector cells (carrying a receptor) are attached to a surface, this attachment is optional, and, in the step of applying the force instead a differential force is applied. In yet another embodiment, in methods of the invention wherein either the target cells or effector cells are attached to a surface, this attachment is optional, and, in the step of applying the force instead a differential force is applied, wherein the differential force is such that cells that are bound via a synapse remain bound to each other and formed aspecific cell-cell bonds are broken.

### EXAMPLES

### Example 1

### Primary Cells and Cell Lines

In the experiment second-generation 41BBζ CAR constructs were used with binding domains derived from anti-CD19 antibodies, clones FMC63 (CAR1), 4G7 (CAR2) and HD37 (CAR3) (Kramer, A M; (2017) Delineating the impact of binding-domain affinity and kinetic properties on Chimeric Antigen Receptor T-cell function. Doctoral thesis, UCL (University College London)). All binding domain molecules of these second-generation 41BBζ CARs were raised against the same target antigen and have been previously assessed with regard to affinity and found to have dissociation constants (KD) in the range of approximately 1-20 nM. Untransduced or CAR retrovirus transduced primary T-cells were used as effector cells, while Nalm6 cells (obtained from ATCC) were used as target cells. All the cells were cultured with RPMI+Glutamax supplemented with 10% heat inactivated fetal bovine serum and Penicillin-Streptomycin. The CAR-T cells were sorted for marker gene expression to reduce sample to sample variability. Expression levels of marker gene were calculated using BD Biosciences™ Quantibrite™ Phycoerythrin (PE) Beads to ensure comparability of samples. Experiments were performed on day 6 post-transduction and in triplicates. On the day of the experiment, the CAR-T cells were harvested, counted and used either for functional cytotoxicity assays or cellular avidity measurements.

### Functional Cytotoxicity

The functional assay was set up using 100,000 target Nalm6 cells and with varying CAR-T cell numbers to make up 1:2 or 1:4 E:T ratios. The readout was by flow cytometry using CountBright™ Absolute Counting Beads, while cytokines were measured using Biolegend ELISA kits as commercially available.

### Avidity Measurement

Z-Movi® chips (obtained and as available from LUMICKS, with channel dimensions of 7×2×0.1 mm and made of glass) were coated the day before performing the experiment with PLL. The following morning the target cells, Nalm6, were seeded in serum-free culture medium, incubated for 0.5 h at 37° C., and the medium was replaced with RPMI complete medium. The target cells were incubated for at least 1.5 h before starting the cellular avidity experiment. The effector cells were stained with CellTrace™ Far Red dye (Thermo Fisher Scientific) at 1 μM for 15 minutes in PBS at 37° C., then resuspended at 10 million/mL in complete medium and used for the avidity experiments. The effector cells (i.e. cells of interest) were introduced in the target cell-seeded flow cell, incubated for 2 or 5 minutes, and then a force ramp was applied throughout for 2.5 minutes using Z-Movi® operated with the Ocean V1.4 software which was customized to allow control of incubation time.

### Results

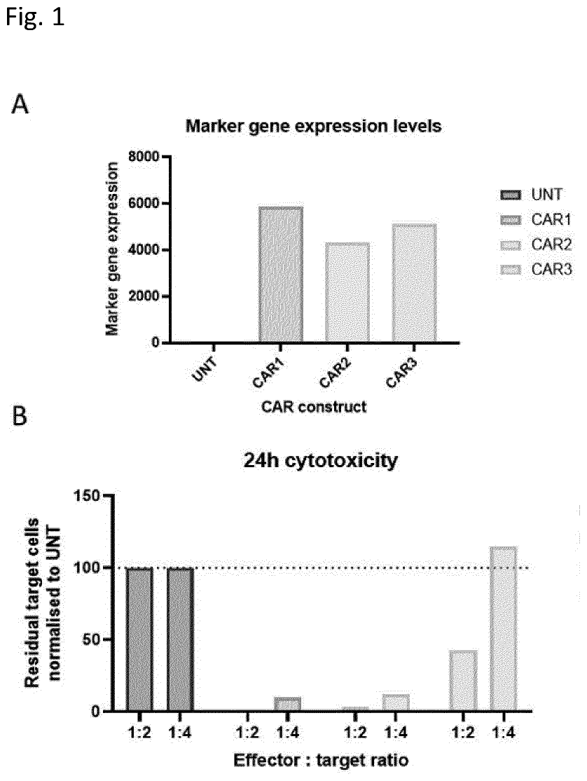

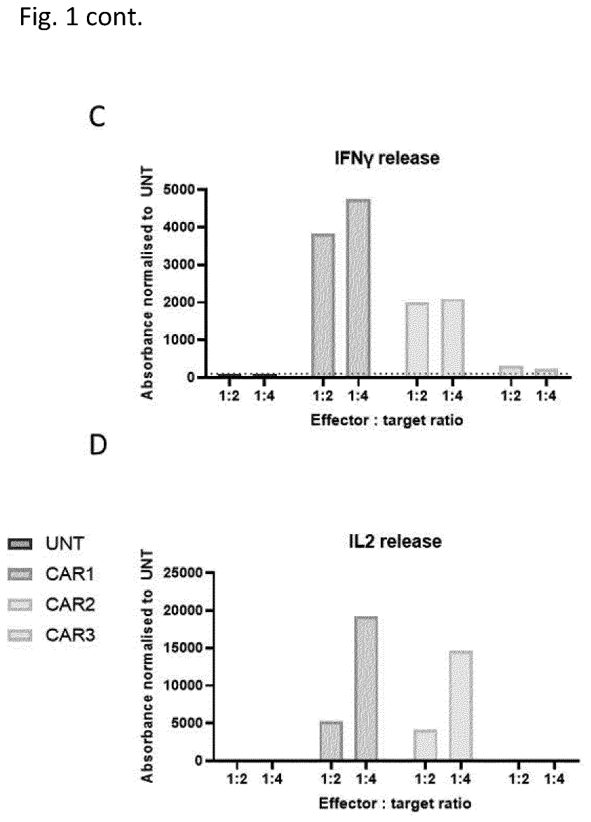

The three CAR-types were transduced and expressed at comparable levels in primary T cells (FIG. 1, A). A 24 h cytotoxicity assay showed that CAR1 and CAR2 killed nearly all the target cells at both 1:2 and 1:4 effector to target ratios. CAR3 underperformed and at 1:4 E:T ratio failed to control the tumor growth (FIG. 1, B). The IL2 and IFNγ secretion were also compared between the CARs following 24 h co-culture (FIGS. 1, C and D). CAR1 resulted in more IFNγ secretion for both E:T ratios than for both CAR2 and CAR3. IL2 secretion was comparable between CAR1 and CAR2, while CAR3 didn't result in any apparent secretion. The results of these in vitro functional assays indicate that CAR3 may not be suitable for further investigations. However, CAR 1 and 2 performed comparable in both cytotoxicity and cytokine assays. Based on these assays alone, one cannot select one over the other for further downstream in vivo testing.

From the cellular avidity measurements, it was observed that following 2 minute on-chip incubation, CAR2 T cells showed greater cellular avidity compared to CAR1 and CAR3 (FIG. 2, A). However, when incubation time was increased to 5 minutes, CAR1 showed similar levels of cellular avidity towards the target cell line as CAR2 (FIG. 2, B). When plotted, CAR1 shows a greater change in avidity over time compared to CAR2. This suggests CAR1 has slower on-rate compared to CAR2. Surprisingly, CAR2 affinity measurement showed it to have a slower on-rate than CAR1.

Following a typical avidity measurement which included 5 minutes on-chip incubation time, CAR1 and CAR2 (aka FMC and 4G7) showed similar cellular avidity. Hence, running cellular avidity experiments using CAR-T cells utilising binding domains derived from molecules of similar affinity did not allow to distinguish between the CARs, at least not between CAR1 and CAR2. By decreasing incubation time to 2 minutes, differences could be observed. However, relying only on the results obtained with either 2 or 5 minutes did give much further insight, other than that with a 2 minute incubation time perhaps CAR1 would be of less interest. However, by combining the results from both 2 and 5 minutes, further insight was provided, as the resulting slope, or relative increase, or also calculated ratio, may be representative of e.g. an on-rate of a CAR-T (see Table 1). For CAR1 and CAR2 the observed plots (or likewise, calculated Delta, Slope or Ratio) may indicate for example that the on-rate for CAR1 is slower than for CAR2.

Combined, the results show it can be challenging to differentiate CAR constructs by their in vitro functional activity alone. Cellular avidity measurements allow to shed light on cell-cell interaction and its effects on CAR-T cell performance, and measuring cellular avidity over time can improve understanding of the binding dynamics between CAR-T cells and target tumor cells. Performing a time-lapse cellular avidity experiment can thus be more informative as opposed to a single cellular avidity experiment.

### Example 2

### Primary Cells and Cell Lines

In the experiment a second-generation 41BBζ CAR construct was used with binding domains derived from anti-CD19 antibodies, clone FMC63 (CAR1) (Kramer, AM; (2017) Delineating the impact of binding-domain affinity and kinetic properties on Chimeric Antigen Receptor T-cell function. Doctoral thesis, UCL (University College London)). The binding domain molecule of this second-generation 41BBζ CAR has been previously assessed with regard to affinity and found to have dissociation constants (KD) in the range of approximately 1-20 nM. Untransduced or CAR retrovirus transduced primary T-cells were used as effector cells, while CD19-expressing MM1S cells (obtained from ATCC and thereafter engineered to express CD19) were used as target cells. All the cells were cultured with RPMI+Glutamax supplemented with 10% heat inactivated fetal bovine serum and penicillin-streptomycin. Experiments were performed on day 6 post-transduction and in duplicate. On the day of the experiment, the CAR-T cells were harvested, counted and used for cellular avidity measurements.

### Avidity Measurement

Z-Movi® chips (obtained and as available from LUMICKS, with channel dimensions of 7×2×0.1 mm and made of glass) were coated the day before performing the experiment with PLL. The following morning the target cells, CD19-expressing MM1S cells, were seeded in serum-free culture medium, incubated for 0.5 h at 37° C., and the medium was replaced with RPMI complete medium. The target cells were incubated for at least 1.5 h before starting the cellular avidity experiment. The effector cells were stained with CellTrace™ Far Red dye (Thermo Fisher Scientific) at 1 μM for 15 minutes in PBS at 37° C., then resuspended at 10 million/mL in complete medium and used for the avidity experiments. The effector cells (i.e. cells of interest) were introduced in the target cell-seeded flow cell, incubated for 2, 5 or 10 minutes, and then a force ramp was applied throughout for 2.5 minutes using z-Movi© operated with the Ocean V1.4 software which was customized to allow control of incubation time.

### Results

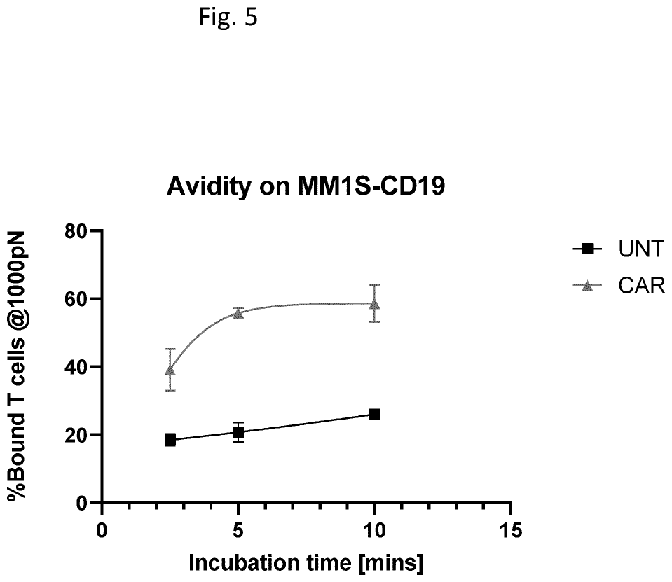

From the cellular avidity measurement, it was observed that following 2.5 minute on-chip incubation, CAR1 T cells showed greater cellular avidity compared to untransduced (see FIG. 5; about 40% bound cells compared to about 20% bound cells for untransduced). When incubation time was increased to 5 minutes, CAR1 showed higher levels of cellular avidity towards the target cell line compared to untransduced cells than at an incubation of 2.5 minutes (see FIG. 5; about 55% bound cells compared to about 20% bound cells for untransduced). When incubation time was increased to 10 minutes, CAR1 showed similar to slightly lower levels of cellular avidity towards the target cell line compared to untransduced cells than at an incubation of 5 minutes (see FIG. 5; about 55% bound cells compared to about 25% bound cells for untransduced).

The results show that performing a time-lapse cellular avidity experiment can be more informative as opposed to a single cellular avidity experiment. The results clearly present a further parameter, i.e. they show that a plateau is reached for the cellular avidity score determined. The results demonstrate that at an incubation time of 5 minutes and onwards the cellular avidity score that is determined no longer substantially increases. Hence, by determining multiple cellular avidity scores and plotting them against time, the plateau of the plot is determined, showing among others the cellular avidity score of the plateau and the incubation time when this plateau is substantially reached.

TABLE 1

| | CA | CA | | Slope | |
|---|---|---|---|---|---|
| | (2 min) | (5 min) | Delta | CA | Ratio |
| | (%) | (%) | CA (%) | (%/s) | CA |
| UNT | 0.162 | 10.9 | 10.7 | 0.060 | 67.3 |
| CAR1 | 18.6 | 79.5 | 60.9 | 0.338 | 4.27 |
| CAR2 | 43.1 | 82.4 | 39.3 | 0.218 | 1.91 |
| CAR3 | 9.75 | 21.4 | 11.6 | 0.065 | 2.19 |

1. A method of assessing cellular avidity of an effector cell carrying a receptor, and target cells comprising the steps of:
a) providing target cells;
b) providing effector cells carrying a receptor;
wherein the target cells or the effector cells carrying a receptor are attached to a surface;
c) contacting the effector cells carrying a receptor with the target cells to allow the effector cells carrying a receptor to interact with the target cells, wherein the interaction is for a first defined time;
d) applying a force, wherein the force is in a direction away from the attached cells, such that at least part of the cells bound to the cells attached to the surface move away therefrom;
e) detecting effector cells carrying a receptor that have remained bound with the target cells and attached to the surface after applying the force, and provide a cellular avidity score;
f) perform steps c), d) and e) with a second defined time for step c).

2. A method of assessing cellular avidity of an effector cell carrying a receptor, comprising the steps of:
a) providing target cells attached to a surface;
b) providing effector cells carrying a receptor;
c) contacting effector cells carrying a receptor with the target cells to allow the effector cells carrying a receptor to interact with the target cells, wherein the interaction is for a first defined time;
d) applying a force on the effector cells carrying a receptor, wherein the force is in a direction away from the target cells;
e) determine effector cells carrying a receptor that have detached and/or remain bound in step d) and provide a cellular avidity score;
f) perform steps c), d) and e) with a second defined time for step c).

3. Method in accordance with claim 1, wherein steps c), d) and e) of the method is performed with a third or more defined times.

4. Method in accordance with claim 1, wherein the defined times are selected from 1 minute to 10 minutes.

5. Method in accordance with claim 1, wherein the cellular avidity scores determined are plotted against time and the slope of the plot is determined.

6. Method in accordance with claim 1, wherein the cellular avidity scores determined are plotted against time and the plateau of the plot is determined.

7. Method in accordance with claim 1, wherein the cellular avidity score at the first and second defined time is determined at a defined force.

8. Method in accordance with claim 1, wherein effector cells with different receptors are provided and for each effector cell with a receptor the method is performed and results compared.

9. Method in accordance with claim 8, wherein the effector cells with different receptors are ranked based on slope and/or plateau.

10. Method in accordance with claim 1, wherein of effector cells carrying a receptor bound with target cells after the step of applying the force the presence of a marker associated with synapse formation is determined.

11. Method in accordance with 10, wherein cellular avidity scores are determined based on the number of effector cells carrying a receptor with the marker associated with synapse formation.

12. The method in accordance with claim 1, wherein after the step of applying the force, the cells are resuspended and a differential force is applied such that formed aspecific cell-cell bonds are broken.

13. The method in accordance with claim 1, wherein cells are optionally attached to a surface, and wherein in the step of applying a force away from the attached cells, instead a differential force is applied.

14. The method in accordance with claim 13, and the differential force is such that cells that are bound via a synapse remain bound to each other and formed aspecific cell-cell bonds are broken.
